(19) United States
(12) Patent Application Publication
(10) Pub. No.: US 2024/0037369 A1
(43) Pub. Date: Feb. 1, 2024

(54) METHOD AND APPARATUS FOR MULTIPLE-BEAT DETECTION USING ELECTROCARDIOGRAM GLOBAL FEATURE VECTORS

(71) Applicant: SEERSTECHNOLOGY CO.,LTD., Seongnam-si, Gyeonggi-do (KR)

(72) Inventors: Youngshin LEE, Yongin-si, Gyeonggi-do (KR); Heeseok SONG, Hwaseong-si, Gyeonggi-do (KR); Yunkwan KIM, Incheon (KR)

(73) Assignee: SEERSTECHNOLOGY CO.,LTD., Seongnam-si, Gyeonggi-do (KR)

(21) Appl. No.: 18/274,723

(22) PCT Filed: Dec. 23, 2021
(86) PCT No.: PCT/KR2021/019751

§ 371 Date: Jul. 28, 2023

(30) Foreign Application Priority Data
Dec. 21, 2021 (KR) 10-2021-0184201

(51) Int. Cl. G06N 3/0442

(52) U.S. Cl. CPC G06N 3/0442

(57) ABSTRACT

Disclosed are a method and an apparatus for multiple-beat detection using electrocardiogram global feature vectors. This method and apparatus extracts global features of each electrocardiogram wave, and extracts and learns, using the extracted global features as input vectors, a pattern of global features of a consecutive electrocardiogram wave by applying an attention mechanism to a weighted feature matrix in consideration of the degree of contribution of each feature to detect multiple beats.

### TECHNICAL FIELD

The present invention relates to a method and an apparatus for multiple-beat detection using electrocardiogram global feature vectors.

### BACKGROUND ART

The content described below merely provide background information related to the present invention and does not constitute a prior art.

General algorithms for arrhythmia detection and classification have been developed in various forms.

A convolutional neural network (CNN)-based algorithm refers to an algorithm for learning only local information with reference to only one wave. The CNN algorithm shows high performance of over 95% in various evaluation scales, but in the case of abnormal waves that appear consecutively in a similar form to normal waves, consecutive and global patterns were not considered, which results in performance degradation.

In the CNN algorithm, in configuring consecutive data as an input and proceeding with CNN-based training and inference, since only local features are extracted and only the corresponding patterns are learned, problems occur in learning a consecutive relationship.

An LSTM (Long Short-Term Memory)-based algorithm has been proposed to overcome the problem related to the learning of the consecutive relationship in the CNN-based algorithm. However, in the LSTM algorithm, in a case where an input length is long and related elements are far apart, it is difficult to learn the consecutive relationship between respective samples due to long-term dependency of the respective samples in an input electrocardiogram.

In a case where a large number of LSTMs are configured to overcome the above-mentioned long-term dependency, there is a problem that the speed of learning and inference slows down due to increase in the number of parameters. Conventional deep learning-based beat detection and classification algorithms output only one result for one input, and thus, in a case where multiple beats exist in one input, it is difficult to detect and classify multiple abnormal beats.

### DISCLOSURE

### Technical Problem

The present invention has been made in view of the above problems, and it is an object of the present invention to provide a method and an apparatus for multiple-beat detection using electrocardiogram global feature vectors, capable of extracting global features of each electrocardiogram wave, and extracting and learning, using the extracted global features as input vectors, a pattern of global features of consecutive electrocardiogram waves by applying an attention mechanism to a weighted feature matrix in consideration of the degree of contribution of each feature to detect multiple beats.

### Technical Solution

In accordance with the present invention, the above-mentioned object can be accomplished by the provision of a multiple-beat detecting apparatus including: a multi-input unit that receives an input of a plurality of pieces of heartbeat data from consecutive heartbeat data; a global feature extracting unit that extracts a global feature for each of the plurality of pieces of heartbeat data; an attention block unit that generates encoding attention data by combining position information on the global feature; a bidirectional LSTM unit that outputs bidirectional LSTM result values obtained by performing a bidirectional long short-term memory (LSTM) process on the attention data; and a classification unit that performs classification by checking the position information for respective multiple inputs on the basis of the bidirectional LSTM result values.

### Advantageous Effects

As described above, according to the present invention, it is possible to extract global features of each electrocardiogram wave, and to extract and learn, using the extracted global features as input vectors, a pattern of global features of consecutive electrocardiogram waves by applying an attention mechanism to a weighted feature matrix in consideration of the degree of contribution of each feature to detect multiple beats.

### DESCRIPTION OF DRAWINGS

FIG. 1 is a diagram illustrating bio-signal data processing in the field of bio-signal processing according to an embodiment of the present invention;

FIG. 2 is a diagram illustrating a multiple-beat detecting apparatus according to an embodiment of the present invention;

FIG. 3 is a flowchart illustrating a method for detecting multiple beats using electrocardiogram global feature vectors according to an embodiment of the present invention;

FIG. 4 is a diagram showing a structure of an arrhythmia classification model based on an attention mechanism using an electrocardiogram global feature matrix according to an embodiment of the present invention;

FIG. 5 is a diagram showing a model structure for extracting an attention matrix of an electrocardiogram global feature matrix according to an embodiment of the present embodiment; and

FIG. 6 is a diagram showing a bidirectional LSTM structure for detecting and classifying beats while identifying the continuity of respective attention matrices according to the embodiment of the present invention.

### REFERENCE NUMERALS

200: Multiple-beat detecting apparatus
210: Multi-input unit
220: Global feature extracting unit
230: Attention block unit
240: Bidirectional LSTM unit
250: Classification unit

### BEST MODE

Hereinafter, exemplary embodiments of the present invention will be described in detail with reference to the accompanying drawings.

FIG. 1 is a diagram illustrating bio-signal data processing in the field of bio-signal processing according to an embodiment of the present invention.

A multiple-beat detecting apparatus 200 according to the present embodiment may be applied to one-dimensional (1D) bio-signal data processing in the field of bio-signal processing. The multiple-beat detecting apparatus 200 divides an electrocardiogram wave into a P wave, a QRS-complex (N, S, V), a T wave, and a noise wave included in the electrocardiogram wave in a wave unit.

The multiple-beat detecting apparatus 200 receives an electrocardiogram signal obtained by interpreting electrical activities of the heart recorded by electrodes attached to the skin and equipment outside the body. The multiple-beat detecting apparatus 200 is used for the purpose of diagnosis or research on abnormal activities of the heart, using the electrocardiogram signal for measuring the rate and regularity of heartbeats.

The multiple-beat detecting apparatus 200 detects such arrhythmia that the heart beats irregularly, too quickly, or too late on the basis of the electrocardiogram signal. The multiple-beat detecting apparatus 200 detects and classifies beat-based arrhythmia (for example, premature ventricular contraction, premature atrial contraction, or ectopic beat).

A general beat classification algorithm determines a beat by considering only one beat pattern, but it is difficult to distinguish, for example, a ventricular abnormal beat from a normal beat on the basis of only one beat pattern. Since such a general beat classification algorithm does not consider consecutive patterns even in the case of abnormal beats that appear consecutively, it is difficult to perform accurate classification.

The multiple-beat detecting apparatus 200 according to the present embodiment includes a multi-input unit for enabling input of a plurality of beats. The multiple-beat detecting apparatus 200 accurately classifies abnormal beats, normal beats, and consecutive abnormal beats in consideration of the characteristics and consecutive patterns of multiple beats. The multiple-beat detecting apparatus 200 calculates the number of occurrences of normal beats, the number of occurrences of abnormal beats, and the number of consecutive abnormal beats on the basis of the characteristics and consecutive patterns of the multiple beats.

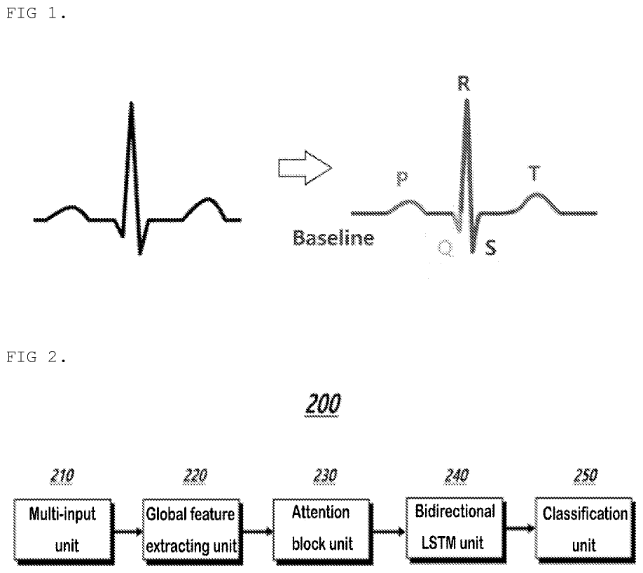

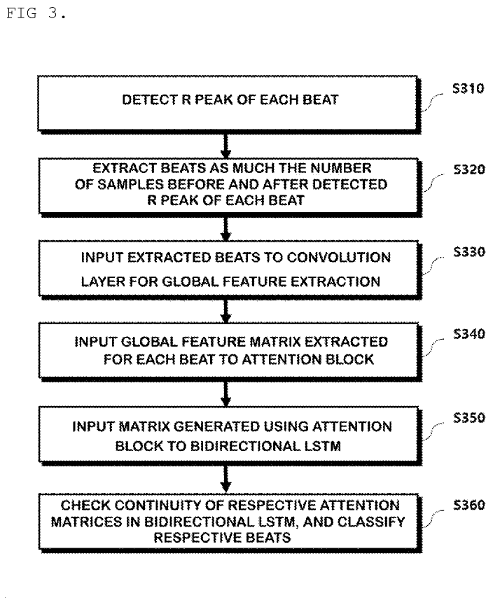

FIG. 2 is a diagram illustrating a multiple-beat detecting apparatus according to an embodiment of the present invention.

The multiple-beat detecting apparatus 200 according to the present embodiment includes a multi-input unit 210, a global feature extracting unit 220, an attention block unit 230, a bidirectional LSTM unit 240, and a classification unit 250. Here, components included in the multiple-beat detecting apparatus 200 are not necessarily limited thereto. The respective components included in the multiple-beat detecting apparatus 200 are connected to a communication path that connects software modules or hardware modules inside the apparatus and are able to communicate with each other. These components communicate with each other using one or more communication buses or signal lines.

Each component of the multiple-beat detecting apparatus 200 shown in FIG. 2 means a unit that processes at least one function or operation, and may be implemented as a software module, a hardware module, or a combination of software and hardware.

The multi-input unit 210 receives an input of consecutive heartbeat data. Here, the multi-input unit 210 receives an input of consecutive heartbeat data including R peak point information. Further, the multi-input unit 210 receives an input of position information on the beginning and end of the consecutive heartbeat data.

The multi-input unit 210 extracts each piece of heartbeat data corresponding to a certain length before and after an R peak of each heartbeat in the consecutive heartbeat data. The multi-input unit 210 receives an input of each piece of heartbeat data. The multi-input unit 210 receives an input of a plurality of pieces of heartbeat data from the consecutive heartbeat data.

The multi-input unit 210 checks an R peak for an R wave from the consecutive heartbeat data, and extracts an expected length of beats as much as a predetermined number of samples before and after the R peak as the plurality of pieces of heartbeat data.

The global feature extracting unit 220 places a convolution layer in each part of the multi-input unit 210. The global feature extracting unit 220 extracts a global feature matrix that has passed through the convolutional layer placed in each part of the multi-input unit 210.

The global feature extracting unit 220 extracts a global feature for each of the plurality of pieces of heartbeat data. The global feature extracting unit 220 extracts a global feature matrix corresponding to the global features by performing linear projection on the plurality of pieces of heartbeat data.

The attention block unit 230 generates encoding attention data by combining position information on the global features. The attention block unit 230 performs an operation on a matrix obtained by combining global feature vectors and position information vectors for the global feature matrix with different weight parameters to calculate values of Query, Key and Value.

The attention block unit 230 performs a scaled dot-product attention using the values of Query, Key and Value, computes Query-Key transpose values, and then performs a softmax operation on values obtained by dividing the result by a root value of a Key vector dimension to generate an attention matrix corresponding to the attention data.

The attention block unit 230 performs a multi-head attention of merging attention matrices obtained by performing the scaled dot-product attention. The attention block unit 230 adds the global feature vectors used to perform a self-attention and a matrix calculated after performing the multi-head attention, and performs normalization to generate the attention matrix.

The attention block unit 230 generates the attention matrix using a multi-head attention block, a first Add & Norm block, a Feed Forward block, and a second Add & Norm block.

The attention block unit 230 concatenates the attention matrices corresponding to multiple inputs, and inputs the result to the bidirectional LSTM unit.

The attention block unit 230 calculates positional encoding using [Equation 1] and [Equation 2]. The attention block unit 230 uses a sine function and a cosine function using ‘pos’ representing positions of feature vectors and ‘i’ values representing dimension information of the feature vectors. The attention block unit 230 calculates a final positional encoding value using hyperparameters of ‘d_{model}’

$${PE}_{( pos , 2 i )} = sin ( pos / {10000}^{\frac{2 i}{{d}_{model}}} ) \qquad [ Equation 1 ]$$

$${PE}_{( pos , 2 i + 1 )} = sin ( pos / {10000}^{\frac{2 i}{{d}_{model}}} ) \qquad [ Equation 2 ]$$

The attention block unit 230 performs a self-attention.

The attention block unit 230 performs an operation on a matrix obtained by combining global feature vectors and position information vectors with different weight parameters to calculate values of Query, Key and Value.

As expressed in [Equation 3], the attention block unit 230 performs a scaled dot-product attention using the values of Query, Key, and Value. The attention block unit 230 computes Query-Key transpose values, and then divides the result by a root value of a Key vector dimension. The attention block unit 230 calculates an attention matrix by performing a softmax operation on values obtained by the division.

As expressed in [Equation 4] and [Equation 5], the attention block unit 230 performs a multi-head attention of merging attention matrices obtained by performing the scaled dot-product attention.

The attention block unit 230 first adds the global feature vectors used to perform the self-attention and the matrix calculated after performing the multi-head attention, and then, performs normalization.

Then, the attention block unit 230 inputs the result to a position-wise feed forward neural network to calculate an output matrix.

Attention(Q,K,V)=softmax(QK_{T}/√dₖV) [Equation 3]

MultiHead(Q,K,V)=Concat(head₁, . . . ,headₕ) [Equation 4]

headᵢ=Attention(QWᵢ^{Q},KWᵢ^{K},VWᵢ^{V}) [Equation 5]

The bidirectional LSTM unit 240 outputs bidirectional LSTM result values obtained by performing a bidirectional long short-term memory (LSTM) process on attention data. The bidirectional LSTM unit 240 merges the calculated attention matrices, and then, performs the bidirectional LSTM process to determine long-term dependency and correlation of respective sequence steps.

The bidirectional LSTM unit 240 extracts features of changes between temporally neighboring next beats while forwarding the bidirectional LSTM result values for the respective multiple inputs. The bidirectional LSTM unit 240 extracts features of changes and errors between temporally neighboring previous beats while backwarding the bidirectional LSTM result values for the respective multiple inputs, to thereby generate the bidirectional LSTM result values.

The classification unit 250 derives the values calculated from the bidirectional LSTM unit 240, and outputs classification information. In other words, the classification unit 250 performs classification by checking position information for the respective multiple inputs on the basis of the bidirectional LSTM result values

The classification unit 250 classifies the bidirectional LSTM result values into one of N (normal beat), S (supraventricular ectopic beat), V (ventricular ectopic beat), F (fusion beat), and Q (unknown beat).

FIG. 3 is a flowchart illustrating a method for detecting multiple beats using electrocardiogram global feature vectors according to an embodiment of the present invention.

The multiple-beat detecting apparatus 200 detects an R peak of each beat included in an electrocardiogram signal (S310). The multiple-beat detecting apparatus 200 extracts beats as much as the number of samples before and after the detected R peak of each beat (S320).

The multiple-beat detecting apparatus 200 inputs the extracted beats to a convolution layer for global feature extraction (S330). The multiple-beat detecting apparatus 200 inputs a global feature matrix extracted for each beat to an attention block (S340).

The multiple-beat detecting apparatus 200 inputs a matrix generated using the attention block to the bidirectional LSTM unit (S350). The multiple-beat detecting apparatus 200 checks the continuity of respective attention matrices in the bidirectional LSTM unit, and detects and classifies the respective beats (S360).

In FIG. 3, an example in which steps S310 to S360 are sequentially executed is shown, the present invention is not limited thereto. In other words, FIG. 3 does not limit a time-series sequence, and thus, for example, the execution sequence of the steps shown in FIG. 3 may be changed, or a plurality of the steps may be executed in parallel.

As described above, the multiple-beat detection method using the electrocardiogram global feature vectors according to the present embodiment shown in FIG. 3 may be implemented as a program, and may be recorded on a computer-readable recording medium. The computer-readable recording medium on which the program for implementing the multiple-beat detection method using the electrocardiogram global feature vectors according to this embodiment is recorded includes all types of recording devices for storing data that is readable by a computer system.

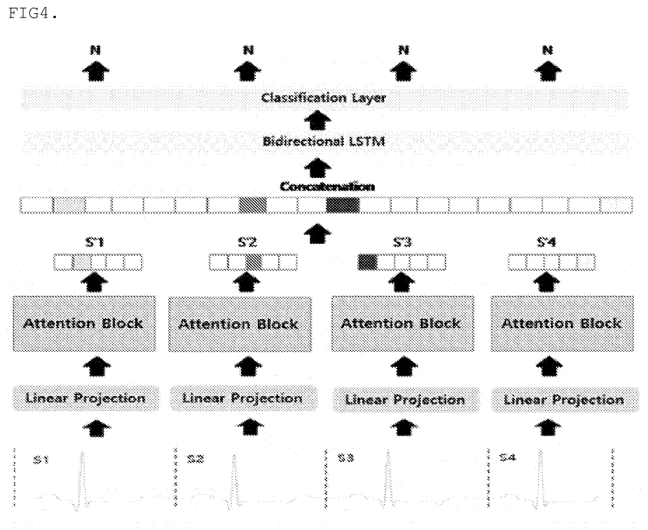

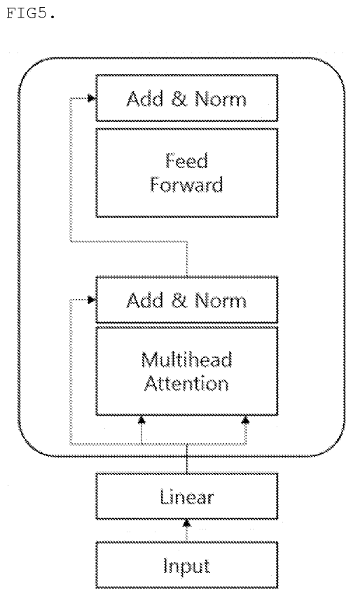

FIG. 4 is a diagram showing a structure of an arrhythmia classification model based on an attention mechanism using an electrocardiogram global feature matrix according to an embodiment of the present invention.

The multiple-beat detecting apparatus 200 recognizes consecutive patterns using respective characteristics of a plurality of beats, and accurately classifies the results. The multiple-beat detecting apparatus 200 calculates the number of occurrences of the classified beats.

The multiple-beat detecting apparatus 200 according to the present embodiment has the following differences from conventional algorithm structures.

\textcircled{1} The multiple-beat detecting apparatus 200 has a multi-input structure.

\textcircled{2} The multiple-beat detecting apparatus 200 extracts global features using data of respective beats passed through the multi-input unit.

\textcircled{3} The multiple-beat detecting apparatus 200 performs an attention block operation using a transformer structure on the extracted global features to calculate a weight of the degree of contribution of each global feature, and extracts a weighted global feature matrix in which the weights are reflected.

\textcircled{4} The multiple-beat detecting apparatus 200 merges the respectively generated and weighted feature matrices, and determines the continuity of the merged matrices using the bidirectional LSTM.

\textcircled{5} The multiple-beat detecting apparatus 200 respectively classifies the feature matrices of which the continuity is determined using originally input position information.

The multiple-beat detecting apparatus 200 extracts an R peak among a P wave, a QRS-complex (N, S, V), and a T wave included in an electrocardiogram wave. The multiple-beat detecting apparatus 200 extracts beats corresponding to a preset margin before and after the R peak. The multiple-beat detecting apparatus 200 extracts signal values corresponding to an expected length of beats as much as a predetermined number of samples before and after the R peak. The multiple-beat detecting apparatus 200 extracts n beats in the same margin before and after the R peak in the electrocardiogram wave, and inputs the n beats as multiple inputs (e.g., S1, S2, S3, S4, . . . , and Sn).

The multiple-beat detecting apparatus 200 places a convolutional network for the respective multiple inputs (S1, S2, S3, S4, . . . , and Sn) to extract global features. The multiple-beat detecting apparatus 200 outputs result values obtained by applying linear projection for the respective multiple inputs (S1, S2, S3, S4 . . . , and Sn). The multiple-beat detecting apparatus 200 extracts the global features from the result values obtained by applying the linear projection using attention blocks.

In a case where S1 is input, the multiple-beat detecting apparatus 200 applies the linear projection and the attention block on S1 to transform S1 into S′1 with a global feature emphasized, and outputs the result. In other words, the multiple-beat detecting apparatus 200 performs multiplication of a weight of the feature, and outputs the result as a matrix. The multiple-beat detecting apparatus 200 concatenates representative features.

The multiple-beat detecting apparatus 200 performs the bidirectional LSTM process on the concatenated representative features. The multiple-beat detecting apparatus 200 arranges the matrices in a row, and then, checks their temporal relationships.

The multiple-beat detecting apparatus 200 performs classification for the multiple inputs on the basis of the bidirectional LSTM result values. In this case, the multiple-beat detecting apparatus 200 confirms position points on the basis of the bidirectional LSTM result values to perform the classification for the respective multiple inputs. The multiple-beat detecting apparatus 200 classifies beats into N (Normal beat), S (Supraventricular ectopic beat), V (Ventricular ectopic beat), F (Fusion beat), and Q (Unknown beat).

FIG. 5 is a diagram showing a model structure for extracting an attention matrix of an electrocardiogram global feature matrix according to an embodiment of the present invention.

The model structure for extracting the attention matrix includes a multi-head attention block, a first Add & Norm block, a Feed Forward block, and a second Add & Norm block.

The multi-head attention block generates an N×M matrix on the basis of values obtained by converting a 1×N matrix corresponding to beat data into an M×1 matrix. For example, in a case where a plurality of 1×60 matrices are input, the multi-head attention block converts the plurality of 1×60 matrices into 60×1 matrices through linear projection blocks. The multi-head attention block creates a 60×60 matrix on the basis of the 1×60 matrix and the 60×1 matrix.

The first Add & Norm block performs normalization in a state where a 1×N matrix is added to the N×M matrix to prevent loss of information. For example, the first Add & Norm block performs normalization in a state where an input value (1×60 matrix) is added to a 60×60 matrix to prevent loss of information.

The Feed Forward Block generates a feature matrix by extracting feature values from values obtained by the normalization. In other words, the feed forward block generates an attention matrix for emphasizing features from the values obtained by the normalization.

The second Add & Norm block performs normalization in a state where the normalized N×M matrix is added to the attention matrix to prevent loss of information, and outputs the attention matrix as a 1×N matrix. For example, the second Add & Norm block performs normalization in a state where input values (normalized 60×60 matrix) are added to the feature matrix to prevent loss of information, and outputs the result values as a 1×60 matrix.

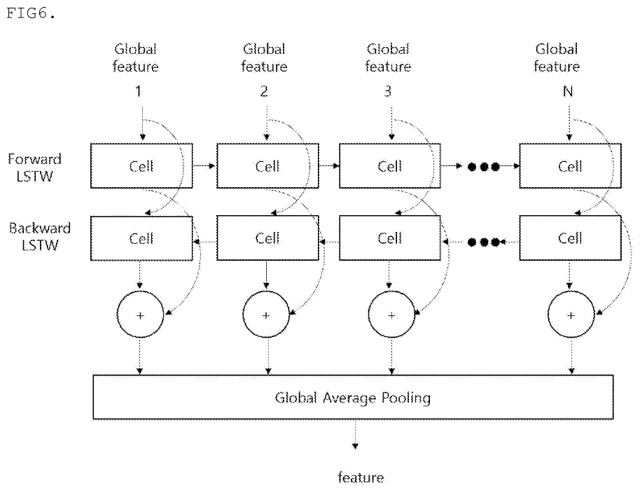

FIG. 6 is a diagram showing a bidirectional LSTM structure for detecting and classifying beats while checking the continuity of respective attention matrices according to the present embodiment.

The multiple-beat detecting apparatus 200 may be extended to a model that predicts the next wave. The multiple-beat detecting apparatus 200 may be extended to a clinical decision support system in which a pre-learned encoder and other deep learning-based models are combined. The multiple-beat detecting apparatus 200 may be extended to a provisional classification category.

The multiple-beat detecting apparatus 200 may learn a correlation between respective heartbeats by applying various attention methods between global features of the respective heartbeats on the basis of an attention mechanism that uses the global features of the respective heartbeats as inputs.

The multiple-beat detecting apparatus 200 may increase accuracy compared to conventional arrhythmia detection and classification algorithms by learning positions where beats are concentrated in consecutive data together with the correlation learning.

The multiple-beat detecting apparatus 200 may be used as a model for predicting the next wave in performing transfer learning using only an input unit and an encoding unit without a classification unit after pre-training. The multiple-beat detecting apparatus 200 may be used as a clinical decision support system in a case where output values of the encoding unit are used in another deep learning-based model.

The bidirectional LSTM unit 240 of the multiple-beat detecting apparatus 200 checks correlations between cell values (sets) as input values (S1, S2, S3, S4, . . . , Sn), after going through the forwarding and backwarding processes, and finally determine their labels.

The bidirectional LSTM unit 240 of the multiple-beat detecting apparatus 200 extracts features of changes between temporally neighboring next beats while forwarding the cell values (sets) as the input values (S1, S2, S3, S4, . . . , Sn). The bidirectional LSTM unit 240 of the multiple-beat detecting apparatus 200 extracts features of changes and errors between temporally neighboring previous beats while backwarding the cell values (sets) as the input values (S1, S2, S3, S4 . . . Sn).

The above description is merely an example of the technical idea of the present invention, and various modifications and variations may be made by those skilled in the art without departing from the concept of the present invention. Therefore, the present embodiments are not intended to limit the technical idea of the present invention, and the scope of the technical idea of the present invention is not limited by these embodiments. The scope of protection of the present invention should be construed according to claims, and all technical ideas within the scope equivalent thereto should be construed as being included in the scope of the present invention.

1. A multiple-beat detecting apparatus comprising:
a multi-input unit that receives an input of a plurality of pieces of heartbeat data from consecutive heartbeat data;
a global feature extracting unit that extracts a global feature for each of the plurality of pieces of heartbeat data;
an attention block unit that generates encoding attention data by combining position information on the global feature;
a bidirectional LSTM unit that outputs bidirectional LSTM result values obtained by performing a bidirectional long short-term memory (LSTM) process on the attention data; and
a classification unit that performs classification by checking the position information for respective multiple inputs on the basis of the bidirectional LSTM result values.

2. The apparatus according to claim 1, wherein the multi-input unit checks an R peak for an R wave from the consecutive heartbeat data, and extracts an expected length of beats as much as a predetermined number of samples before and after the R peak as the plurality of pieces of heartbeat data.

3. The apparatus according to claim 1, wherein the global feature extracting unit performs linear projection on the plurality of pieces of beat data to extract a global feature matrix corresponding to the global features.

4. The apparatus according to claim 3, wherein the attention block unit performs an operation on a matrix obtained by combining global feature vectors for the global feature matrix and position information vectors with different weight parameters to calculate values of Query, Key, and Value.

5. The apparatus according to claim 4, wherein the attention block unit performs a scaled dot-product attention using the values of Query, Key, and Value to compute Query-Key transpose values, and then performs a softmax operation on values obtained by dividing the result by a root value of a Key vector dimension to generate an attention matrix corresponding to the attention data.

6. The apparatus according to claim 5, wherein the attention block unit performs a multi-head attention of merging the attention matrices obtained by performing the scaled dot-product attention, adds the global feature vectors used to perform a self-attention and a matrix calculated after performing the multi-head attention, and performs normalization to generate the attention matrix.

7. The apparatus according to claim 5, wherein the attention block unit generates the attention matrix using a multi-head attention block, a first Add & Norm block, a Feed Forward Block, and a second Add & Norm block.

8. The apparatus according to claim 7, wherein the multi-head attention block generates an N×M matrix on the basis of values obtained by converting a 1×N matrix corresponding to the heartbeat data into an M×1 matrix.

9. The apparatus according to claim 8, wherein the first Add & Norm block performs normalization in a state where the 1×N matrix is added to the N×M matrix to prevent loss of information.

10. The apparatus according to claim 9, wherein the Feed Forward Block generates the attention matrix for emphasizing the features from values obtained by the normalization.

11. The apparatus according to claim 10, wherein the second Add & Norm block performs normalization in a state where the normalized N×M matrix is added to the attention matrix to prevent loss of information, and outputs the attention matrix to the 1×N matrix.

12. The apparatus according to claim 5, wherein the attention block unit concatenates the attention matrices corresponding to multiple inputs, and inputs the result to the bidirectional LSTM unit.

13. The apparatus according to claim 5, wherein the bidirectional LSTM unit generates the bidirectional LSTM result values by extracting features of changes between temporally neighboring next beats while forwarding the bidirectional LSTM result values for the respective multiple inputs, and extracting features of the changes and errors between temporally neighboring previous beats while backwarding the bidirectional LSTM result values for the respective multiple inputs.

14. The apparatus according to claim 1, wherein the classification unit classifies the bidirectional LSTM result values into one of N (Normal beat), S (Supraventricular ectopic beat), V (Ventricular ectopic beat), F (Fusion beat), and Q (Unknown beat).
